(19) United States
(12) Patent Application Publication
(10) Pub. No.: US 2026/0014286 A1
(43) Pub. Date: Jan. 15, 2026

(54) METHOD AND FACILITY FOR TREATING HUMAN OR ANIMAL TISSUE BY DYNAMICALLY CIRCULATING AN ADDITIVE-CONTAINING SUPERCRITICAL FLUID

(71) Applicants: BIOBANK, LIEUSAINT (FR); CY CERGY PARIS UNIVERSITE, CERGY (FR)

(72) Inventors: Raphaël BARDONNET, SEINE-PORT (FR); Solène ROTA, ERMONT (FR); Emmanuel PAUTHE, CERGY (FR); Michel BOISSIERE, PERSAN (FR)

(73) Assignees: BIOBANK, LIEUSAINT (FR); CY CERGY PARIS UNIVERSITE, CERGY (FR)

(21) Appl. No.: 18/994,100

(22) PCT Filed: Jul. 3, 2023
(86) PCT No.: PCT/FR2023/051010

§ 371 Date: Jan. 14, 2025

(30) Foreign Application Priority Data
Jul. 18, 2022 (FR) FR2207325

(51) Int. Cl. A61L 2/00; A61L 27/36; B08B 7/00

(52) U.S. Cl. CPC A61L 2/0082; A61L 27/3687; B08B 7/0021; A61L 2202/121; A61L 2202/122; A61L 2202/123; A61L 2202/15; A61L 2202/17; A61L 2202/21

(57) ABSTRACT

Collagen-based tissue matrices are treated in a reactor by a flow of supercritical carbon dioxide, including at least one chemical additive inserted via an insertion device that is in communication with a loop. The additive is injected into the liquid or supercritical carbon dioxide when the reactor is already pressurized. The method combines supercritical CO₂ as a solvent and a chemical additive as a co-solvent, in a dynamic treatment flow which circulates in the loop associated with the reactor, in order to increase their action on the treated tissue. The circulation of an additive, which is progressively injected and then recirculated with the CO₂, forms part of a first treatment cycle. Several cycles, each with an additive, may follow one after the other, separated by a step of separating out the additive and the CO₂, which is carried out with or without maintaining the pressurization and the circulation of CO₂.

### TECHNICAL FIELD

This disclosure relates to the field of tissue treatment, for example for tissue implantable during surgical operations. It relates more particularly to a method for cleaning, purifying and/or decontaminating tissue of human or animal origin, using dynamic circulation of an additive-containing supercritical fluid, using a supercritical fluid such as carbon dioxide associated with at least one reactive additive in order to contribute to purifying the tissue matrix. Also disclosed is a system provided with a reactor and a loop for the circulation of additive-containing supercritical fluid in order to implement this method.

### TECHNOLOGICAL BACKGROUND

Document FR 2735372 (or the equivalent patent U.S. Pat. No. 5,723,012) discloses the use of carbon dioxide CO₂ in the supercritical state as a decontaminant in the treatment of a tissue having a collagenous network (i.e. bone, tendon, cartilaginous or ligamentous tissue). This fluid in the supercritical state has an excellent ability to penetrate the porous material of the tissue by diffusion, and helps to inactivate the pathogenic agents that are potentially present.

Treatments with chemically active agents and rinses are carried out in order to obtain a product free of contaminating agents, impurities or organic residues. In practice, the tissue may be immersed in liquid solution containing the active agents. To be effective throughout the entire matrix, some of these agents, corrosive at atmospheric pressure, are used at a concentration that sometimes may be high depending on the chemical treatment. This may result in the disadvantage of degrading the mechanical and biological properties of the matrix. More generally, it remains complicated to optimize the compromise between effectiveness of the chemical treatment for decontamination purposes and effectiveness of the extraction of unwanted residues at the end of the cleaning operations.

There is still room for improvement in obtaining a tissue or tissue matrix that is purified and decontaminated (for example, purged of its organic impurities and any pathogens) and retains good mechanical and biological properties.

### SUMMARY

The present disclosure improves the situation, in particular by seeking to optimize the use of purifying and decontaminating products.

To this end, a method is proposed for treating a tissue of human or animal origin by extracting organic matter residues in order to purify and/or decontaminate said tissue, the method using carbon dioxide in the supercritical state and a reactor provided with an inlet and an outlet, the method comprising the steps consisting essentially of:
arranging the tissue within an internal volume of the reactor;
pressurizing the reactor, by inserting carbon dioxide in the supercritical state into the internal volume via the inlet, such that the carbon dioxide in the supercritical state reaches an area of contact with the tissue (typically at a determined pressure and temperature) and exits the reactor via the outlet;
maintaining a flow of carbon dioxide in the supercritical state passing through the reactor, preferably by opening the outlet of the reactor using a valve regulating the pressure of the internal volume of the reactor at a desired value;
adding, for example upstream of the reactor, at least one chemical additive for purification and/or decontamination into the flow of liquid or supercritical carbon dioxide being circulated so as to reach the area of contact, in order to create a circulation of a treatment flow within the internal volume which combines the carbon dioxide in the supercritical state as an extraction solvent and the at least one chemical additive as an extraction co-solvent, the concentration of co-solvent being determined according to its solubility in carbon dioxide in the supercritical state;
while maintaining the pressurization, causing the treatment flow to circulate, outside of the area of contact, in a loop that is in communication with the reactor inlet, thereby creating a recirculation which contributes to making the treatment flow dynamic, allowing the co-solvent to penetrate into the tissue matrix (therefore contributing to purifying and/or decontaminating the tissue) and, preferably, while allowing the extraction and separation of a portion of the residues in the loop (such a separation being achievable for example after the phase of treatment with the additive).

The method may include one or more treatment cycles, each using a co-solvent. The additive used as co-solvent is typically liquid, in particular liquid at ambient conditions. After each treatment cycle, at least one of the following arrangements may be used:
the separation stage at the reactor outlet may be activated to allow purging the chemical additive for purification/decontamination and some of the residues coming from the internal volume, which are liquid and/or solid, the recirculation of carbon dioxide being maintained in the circuit.
after depressurization of the reactor, a purging of the additive and the residues remaining in the internal volume may be activated.
the reactor and the loop may be depressurized and compressed air may be injected into the reactor and the loop in order to discharge, through an outlet placed downstream of the reactor, the chemical additive for purification/decontamination, and some of the residues remaining in the internal volume, which are liquid and/or solid.
a rinse, for example with purified water, may be carried out in the same direction or in a different direction than that in which the treatment flow circulates in the reactor. This can facilitate the elimination of residues that may remain in the internal volume of the reactor. This discharge may be carried out by a separate path than the inlet and outlet used for the flow of supercritical carbon dioxide or may be positioned on a Y-branch before the inlet or after the outlet of the reactor.

The method thus uses a reactor for extracting residues, inside which circulates a dynamic treatment flow joining/passing through a loop, which is a reactor maintained at a pressure and temperature that are respectively higher than the critical pressure and temperature of carbon dioxide, for example above 73 bar and 31° C. (the reactor is thus pressurized; pressures ranging from 100 to 400 bar may be used in certain options). The loop may contribute to the cleaning by retaining, preferably by filtration, residues extracted from the matrix and preventing their recirculation. One advantage of this method is the combined effect of penetration into the tissue matrix, thanks to the supercritical CO₂, and of chemical treatment with a concentration of co-solvent that can be low/reduced, resulting in less degradation of the mechanical and biological properties of the tissue matrix.

In the method implemented in this manner, an action of cleaning/eliminating the residues is obtained, some of the residues being discharged with the dynamic treatment flow. The use of a loop and a dynamic flow tends to limit the accumulation of reagents or residues in the area of contact, with the knowledge that with cycling, the volume of co-solvent inserted can be optimized (the total volume of co-solvent injected can be minimized). In some options, this volume is adjusted according to the amount of tissue matrix to be treated, the volume of the reactor, and the volume of the loop.

In some advantageous options, a sequence of treatments with a first additive then with a second additive (each as a co-solvent of the supercritical CO₂) may be carried out without draining the reactor where the treatment is carried out. A separation stage accessible via a loop bypass may facilitate discharging/separating out the first additive. In other words, in certain cases it is possible to avoid a depressurization (during an intermediate phase, between two treatment cycles) which can be detrimental to the integrity of the tissue.

In the method thus implemented, it is possible to control the effective concentration of reagent in the area of contact with the tissue matrix, by adding additive directly into the supercritical carbon dioxide circulating towards the reactor, for example using a flow rate for the injection of co-solvent that is proportional to the flow rate of the supercritical carbon dioxide.

The chemical reagent(s)/additive(s) are preferably liquid under ambient conditions. Each additive may form a liquid co-solvent (by being supplied in the liquid state from a tank or similar source of additive).

The method is a departure from solutions that rely on static treatment cycles with the action to purify/decontaminate the material being carried out by soaking in a pure or diluted bath of reagent. Using recirculation, it is possible to obtain a cleaning efficiency at a much lower concentration and quantity of active product, with a comparable/identical exposure time.

The method is a departure from solutions that rely on static treatment cycles with the action to decontaminate the material being carried out by impregnation in supercritical carbon dioxide with a co-solvent additive. It is possible to use a larger amount of co-solvent while having the same concentration of co-solvent at the tissue area of contact, with a comparable/identical exposure time. By also allowing the extraction of residues by means of the dynamic flow of supercritical carbon dioxide, it is possible to obtain a higher purification/decontamination efficiency.

In some options, the additive is added to the supercritical carbon dioxide at a volume flow rate ratio of 1/50 or at least a ratio that is between 1/5 and 1/100 (for example between 1/10 and 1/100), which can be useful in avoiding oversaturating the carbon dioxide with co-solvent. The concentration of chemical additive can be very low compared to more conventional treatments, with good results. This facilitates maintaining the integrity of the matrix forming the tissue, ensuring that the biomechanical and biological properties are maintained. The method thus may facilitate the manner of adjusting the supply of decontaminating chemical additive as closely as possible to what is required, for example by not exceeding a concentration threshold of chemically active additive in the supercritical CO₂.

In some embodiments, the method uses a reactor and a set of valves, as well as a filter and/or a separation stage which contribute to separating out the residues, for example by including a filter for separating solids and/or at least one collector. The use of a filter may allow separating solid residues that could interfere with maintaining the looping circulation by blocking a pressure control valve. Such a filter, which prevents/limits the circulation of solid residues further downstream in the loop, can avoid the reintroduction of these residues into the area of contact.

The separation stage may be arranged in a part of the loop where carbon dioxide is circulating in the gaseous or liquid state. The same applies to the filter. Unlike the filter, the separation stage allows the additive and the carbon dioxide to be separated/collected. In some options, the filter and the separation stage are distributed one on either side of a control valve (regulating the pressure in the reactor and located downstream of the reactor outlet). In this case, it may be provided that the separation stage participating in the collection/separation of the co-solvent is located downstream of the control valve and is used only after a treatment cycle which uses a chemical additive (co-solvent).

According to one particular feature of the method, the carbon dioxide circulating in the loop successively transitions to the gaseous state, downstream of a control valve (which may constitute the backpressure regulator, downstream of the reactor outlet), then to the liquid state, by being subjected to a cooling step in a condenser. By the pumping of a circulation pump placed in the loop after the condenser (and upstream of the reactor inlet), it is possible to define a section of the loop in which the carbon dioxide circulates from the pump to the control valve (passing into the reactor), in a supercritical state.

The control valve, mounted on the loop at or shortly after/downstream of the reactor outlet, may consist of a backpressure regulator (or “BPR”) which limits the gas flow leaving the reactor, so as to maintain the desired level of pressurization in the reactor. The backpressure regulator may be used both to circulate carbon dioxide as the sole solvent initially, and then to circulate the treatment flow which combines this solvent with a co-solvent.

The backpressure regulator may be located between the reactor outlet and a separation stage. A high-pressure pump is mounted in the loop, between the separation stage and the reactor inlet, and may be used to circulate the carbon dioxide in the loop and compress it in order to ensure pressurization of the reactor at a given pressure value. An additional pump, also a high-pressure pump, is mounted on a loop bypass or on the reactor, and may be used to insert a chemical additive into the circuit at a flow rate and pressure which are predetermined.

At the outlet of the carbon dioxide circulation pump, the carbon dioxide may be brought to the supercritical state in an exchanger, to raise the temperature of the recirculated flow (via the loop) re-entering the reactor. Pressurization of the reactor is obtained beyond a pressurization threshold, for example higher than the critical pressure of carbon dioxide, the pressurization level possibly being higher than 100 bar, preferably between 140 and 180 bar, for example approximately 160 bar, and by means of the exchanger, the flow of carbon dioxide is brought to a certain temperature, for example higher than the critical temperature of carbon dioxide, preferably between 35 and 50° C., for example 40° C.

Unlike static impregnation, the reactor accepts the fluid in the supercritical state, here CO₂, in phases of dynamic circulation. This enables not only the penetration of the supercritical fluid into the entire tissue matrix, for example bone or cartilaginous tissue, a tendon, or a membrane, but also a replenishment in the area of contact of the additive inserted as a co-solvent. The outlet is optionally axially opposite the inlet when the reactor has a direction of extension or an axis of elongation, for example in a vertical or horizontal direction.

The temperature of the reactor, heated and above 31° C., may be maintained below a threshold that typically is about 50° C., for example with a heated internal volume that is between 33 and 47° C., optionally between 37 and 43° C.

In some embodiments of the method, one or more of the following arrangements may be used:
the treatment by supercritical CO₂ with recirculation may commence without the presence of chemical additive (and thus without any chemical additive present in the reactor, at least for a first treatment step with recirculation).
a step of estimating a parameter representative of the total amount of additive injected into the loop is carried out as a function of the total mass of tissue to be treated that is present in the reactor, independently of the capacity of the reactor and/or independently of the location/distribution of the tissue within the internal volume of the reactor.
the flow rate of a co-solvent pump is adjusted according to the result/parameter estimated in the estimation step and while taking into account the additive concentration in a co-solvent solution containing said additive conveyed in the loop via the co-solvent pump.
the additive is added gradually, for example by taking into account the flow rate of the pump ensuring the circulation of supercritical CO₂ in the loop, in order to allow its dissolution and to distribute it in the reactor and in the loop, for example by choosing a total injection time which is greater than the time taken to traverse the reactor (dwell time), and optionally also greater than the total time taken by the fluid to complete one round trip (passing through the reactor then the loop to return to the reactor inlet). This time may be a characteristic parameter, calculated by using a tracer.
the loop extends outside the reactor, between the reactor outlet and inlet.
the at least one chemical additive for purification/decontamination may be inserted into the loop upstream or downstream of a heat exchanger which heats the flow of carbon dioxide coming from the pump provided in the loop, before it enters the reactor.
the chemical additive is inserted between the outlet of the condenser/cooling means used to liquefy the carbon dioxide and the inlet of the reactor, for example in a section where the carbon dioxide is in the liquid state or in the supercritical state, whether or not the heating by the exchanger or the pressurization by the pump provided in the loop have occurred.
each additive among the at least one additive is added by an additional pump that is separate/distinct from the pump provided in the loop, possibly using a fluidic connection device connected to the loop before the pump.
at least one of the following ratios is predetermined: a first ratio between the total volume of additive delivered by the additional pump and the mass of the tissue to be treated, the first ratio, expressed in ml/g, preferably being between 0.01 and 0.1 or 1, typically between 0.1 and 5, for example between 1 and 3; a second ratio between the volume flow rate delivered by the additional pump and the volume flow rate delivered by the pump provided in the loop, the second ratio preferably being between 1/5 or 1/10 and 1/100, typically between 1/30 and 1/70, for example between 1/45 and 1/55.
each additive among the at least one additive is preferably inserted in liquid form.

The method allows easily controlling the additive injection conditions, which makes it possible to manage the respective concentrations of carbon dioxide and co-solvent relative to the tissue matrix to be treated.

In some preferred embodiment options, the flow rate of supercritical carbon dioxide is between 1 and 100 l/h, for example approximately 100 ml/min for a reactor with an internal volume of 1 liter. For a carbon dioxide flow rate of 100 ml/min, the flow rate of the co-solvent pump is for example between 0.1 and 30 ml/min, possibly between 1 and 10 ml/min depending on the additive.

During the method, establishing the circulation in the loop in order to make the treatment flow dynamic may involve one or more of the following arrangements:
a step of circulating the solvent and co-solvent is carried out in a closed circuit, in the circuit composed of the reactor and the loop, for example by using/opening valves allowing recirculation in the loop and enabling the reactor to remain pressurized.
the treatment flow may initially circulate without co-solvent in the reactor and in the loop, before a step of inserting additive into the closed circuit, for example into the loop or into the reactor.
the reactor is functionally coupled, at the inlet, to a valve for reinserting the flow recirculated via the loop, into the internal volume.
the at least one additive, for example a diluted or undiluted additive coming from a tank, is inserted into a section of the loop where carbon dioxide in the supercritical state is circulating towards the reactor inlet.
the use of a backpressure regulator can make it possible to obtain a very large pressure difference between the reactor (high pressure) and the part of the loop located upstream of the carbon dioxide pump (which is a high-pressure pump).
during the addition, each chemical additive for purification/decontamination is added into an injection line which joins, for example via a fluidic connection coupling, a communicating path in which the carbon dioxide is circulating, such that each chemical additive circulates as a co-solvent of the carbon dioxide in the supercritical state when the latter is circulating in the reactor in order to traverse the area of contact with the tissue.
the injection time for a given additive, several minutes, is optionally less than a circulation period (dynamic circulation) of the closed-loop treatment flow with no new supply of additive.
the communicating path is part of the loop (a loop section).
the method comprises: before said addition, a stabilization phase in which the carbon dioxide circulates in a looping circulation in the circuit formed by the reactor and the loop, while the pressure in the reactor has reached or exceeded a predefined threshold (for example 73 bar) and the temperature has reached a target temperature range (for example above 31° C.), so as to maintain the carbon dioxide in the supercritical state; and during said addition, the looping circulation of the carbon dioxide in this circuit is maintained.

According to one particular feature, the method comprises the implementation, in the pressurized state of the reactor, of at least one or two chemical treatments (in the area of contact, in order to purify the tissue by extracting residues and to decontaminate by inactivating pathogens), which are each carried out in an operating mode of the reactor involving said recirculation, by using the loop. It is possible to circulate the additive in the reactor, from the inlet to the outlet, then in the rest of the circuit (the loop being connected to the inlet and outlet of the reactor) in order to return to the reactor.

During each of these treatments that occur with this operating mode, a chemical additive for purification/decontamination (specific to each of said chemical treatments) may be successively:
inserted/injected, by using a co-solvent pump (and for example with an additive insertion valve connected to the loop, the valve being in an open state), into a section of the loop where the carbon dioxide is circulating in the liquid or supercritical state (typically in the liquid state then in the supercritical state), at a volume flow rate which is for example at least five or ten times lower, optionally a hundred times lower, for example fifty times lower, than that of the carbon dioxide circulating in the loop;
during a supplemental recirculation step without addition, made to recirculate in the reactor and the loop (therefore following a looping circulation without the addition of co-solvent), typically with the additive insertion valve in the closed state.

In some options, the volume flow rate of the additive may for example be greater than or equal to about two percent of the volume flow rate that applies in the loop. One option with a volume flow rate for the insertion (via a co-solvent insertion valve) of the additive that is as low as 2% or less may also be applied for an effective cleaning but requiring more processing time, particularly if the mass of tissue to be treated is large.

Generally, it may be preferred to inject a volume of additive, for example chosen among hydrogen peroxide and a weak acid (peracetic acid PAA for example as an additional additive added in a second treatment cycle after the hydrogen peroxide treatment), which represents a much lower mass of additive than the total mass of tissue placed in the reactor, for example on the order of 25% or less of this mass of tissue.

In the case of a 35% hydrogen peroxide solution (oxygenated water, 35% being percentage by weight), a ratio of approximately 1 ml+/−0.5 ml of solution per 4 g of tissue to be treated may thus be provided, as a non-limiting example. The same applies in the case of an acid solution with disinfectant action, optionally such as an 18% PAA acid solution (peracetic acid diluted to 18% in 99% ethanol), where such a ratio may also apply.

In the following, the injected solution is referred to as co-solvent, which thus may have a certain level of dilution (for example H₂0₂ diluted in H₂O, PAA diluted in ethanol).

In some particular features of the implementation of the method, at least one of the following provisions may be applied:
each chemical additive is recirculated, in the reactor and the loop, before ending the insertion of said additive in the open state of the insertion valve (the injection time of a given additive may be chosen to be longer than the time taken to traverse the reactor, and possibly longer than the time taken to traverse the reactor and the loop, corresponding to one complete round trip).
the reactor may operate with a variable pressure regime with a piston-type device
the reactor may operate with an agitator or an ultrasonic wave device within the internal volume.
after a period of operating in recirculation mode, for example for a sufficient duration corresponding to at least three or four times the time required to traverse the entire circuit, a purge of the reactor and the loop is carried out in order to allow residues to be eliminated.
and before a new treatment step using an additive, a step is planned of recharging with carbon dioxide in the supercritical state in the reactor, with an adjustment of the carbon dioxide flow rate for temperature and pressure conditions which are also predefined.
the two parameters ‘temperature and pressure’ may be 40° C. and 160 bars, possibly with a tolerance of 3% for each of these two values.
between two chemical treatments (treatments combining supercritical carbon dioxide and co-solvent), without moving the tissue and without stopping the pump circulating the carbon dioxide in the reactor and in the loop, the following is provided: a/activate a separation stage connected so as to bypass a section of the loop and accessible by opening a bypass valve (and typically by closing a valve (referred to as the primary valve) located downstream of a control valve);
during the intermediate phase between two chemical treatments, it may also be provided to: b/allow the co-solvent and the remaining residues to accumulate in the separation stage; c/purge the co-solvent and the remaining residues, through valves in the separation stage;
it may also be provided to: d/deactivate the separation stage by closing the bypass valve and opening said primary valve, then restart treatment with a second co-solvent.
between two chemical treatments (among the chemical treatments), and typically without moving the tissue (which remains within the internal volume of the reactor), it is successively provided to: a/depressurize the reactor and the loop (with the knowledge that the pump circulating the carbon dioxide in the reactor and in the loop has been stopped); b/drain the reactor using a suitable drain port (for example a lower port of the reactor which forms said outlet).
the loop is also drained after depressurization.
after draining, it is also planned to: c/purge the reactor and the loop with compressed air in order to remove co-solvent residues. It may also optionally be planned to: d/rinse the reactor with purified water by introducing purified water upstream of the reactor and discharging it downstream of the reactor; the direction of circulation of the purified water may be reversed.
when rinsing is planned, purging with compressed air may be carried out before and/or after step d/. In the absence of rinsing, purging with compressed air may (directly) follow the draining b/.

In an example of a treatment involving several additives, it may be provided that:
the at least one chemical additive for purification and/or decontamination is a first chemical additive, preferably oxygen peroxide, injected into the loop and inserted into the reactor as a co-solvent of carbon dioxide in the supercritical state, in order to carry out a chemical treatment in the area of contact by circulating the first chemical additive in the reactor as well as in the loop which is in communication with the reactor inlet.
in the pressurized state of the reactor and for example after a purge phase, the method comprises the steps consisting essentially of: adding/applying a second chemical additive for purification/decontamination, for example PAA, injected into the loop and inserted into the reactor as a co-solvent of carbon dioxide in the supercritical state.
insertion of the additives may take place without an overlapping/simultaneous presence of the additives in the loop and in the reactor, by carrying out the treatments by the first additive and second additive sequentially. A separation or a purge of the circuit (which includes the reactor) is carried out in the interval between these two treatments.
alternatively, it is possible to provide for forming a heterogeneous flow which simultaneously includes two types of compounds/co-solvents containing chemically active additives for participating in the purification/decontamination of the tissue present in the reactor, during circulation in the loop.

According to one particular feature, the method includes carrying out a chemical treatment in the area of contact by circulating the second chemical additive for purification/decontamination in the reactor as well as in the loop which is in communication with the reactor inlet, the second chemical additive being reinserted into the reactor via the loop as a co-solvent of carbon dioxide in the supercritical state. Typically, the second additive may thus be circulated in a second treatment cycle with an additive-containing flow, after the first additive is removed from the reactor and loop.

According to one option, the method may comprise, after a progressive depressurization which follows said chemical treatment using one or more chemical additives, one or more steps involving an additional agent as a co-solvent. For example, a third additive (ethanol) may be circulated in a third treatment cycle with an additive-containing flow.

The method may include the steps consisting essentially of:
applying an additional agent for decontamination and/or dehydration (in particular ethanol in one embodiment), injected into the loop and inserted into the reactor as a co-solvent of carbon dioxide in the supercritical state; and
carrying out a treatment, in a pressurized state, in the area of contact, resulting from a combined circulation of the additional agent and the carbon dioxide in the supercritical state, in the reactor and in the loop which is in communication with said reactor inlet.

In some implementation options, the additional agent is inserted into the reactor via the loop as a co-solvent of the carbon dioxide in the supercritical state, for example by being inserted through the same valve which served for inserting a first and/or a second additive (one or more additional additives possibly being applied) circulated in the reactor before the selective addition of the additional agent. The additional agent may be a solvent suitable for neutralizing/solubilizing/carrying away an acid used as a chemical additive in the method.

According to one option, a pressure-regulating valve, arranged in the loop downstream of the reactor in the direction of circulation of the treatment flow and upstream of the supercritical carbon dioxide circulation pump, is actuated (for example by varying the degree to which it is opened) in order to activate one or more pressure drops and increases in the reactor during the recirculation, such that the supercritical carbon dioxide and the co-solvent diffuse more deeply inside the tissue matrix, the aim therefore being to render the treatment's action more effective.

According to one aspect of this disclosure, a treatment system is proposed for purifying and decontaminating a tissue (or tissue matrix) of human or animal origin, capable of optimizing the use of additive(s) taking part in the effectiveness of the treatment.

More specifically, a system is proposed for treating a tissue or tissue matrix of human or animal origin and in particular based on collagen, by a flow of supercritical carbon dioxide, in order to implement a treatment method as mentioned above, the system comprising:
a reactor provided with an inlet and an outlet, the reactor delimiting an internal volume for receiving the tissue, the reactor being adapted to be closed and pressurized in order to maintain the supercritical state of the carbon dioxide flow;
a pump and a heating device, designed and arranged to cause the carbon dioxide to transition from the liquid state to the supercritical state, upstream of the inlet of the reactor in the direction of circulation going from the pump to the inlet;
a circuit comprising a loop passing through a point or zone for inserting carbon dioxide in the liquid state, the loop extending between a first loop end connected to the outlet of the reactor and a second loop end connected to the inlet of the reactor, in order to allow recirculation of fluid coming from the reactor, from the outlet to the inlet, the pump being arranged in the loop between the first and second ends and downstream of a condenser;
a connection device for fluidic connection with the loop and associated with additive insertion means, to allow adding at least one chemical additive for purification and/or decontamination of the tissue in a section of the loop located downstream of the condenser and upstream of the inlet of the reactor, such that the fluidic connection device forms a mixer enabling a mixed flow combining the carbon dioxide in the supercritical state as a solvent and the additive as a co-solvent, constituting a treatment flow (coming from the loop) that reaches the tissue within the internal volume of the reactor.

Such a system/facility allows combining a pressurized reactor and a circulation through this reactor, for simultaneously admitting a solvent and a co-solvent forming a dynamic treatment flow for decontamination and extraction of residues, benefitting from the penetration properties of a supercritical fluid such as CO₂. The supercritical fluid may come from a source of liquid carbon dioxide, connected to the loop.

For example, the system may have an opening/closing assembly (provided with valves arranged on the loop), which is adapted to be configured in different states including a first open state, in which a pressure-regulating means or valve (which is for example part of the valves of the opening/closing assembly) allows pressurization of the reactor to be maintained above a threshold (corresponding to a pressure exceeding the critical pressure of carbon dioxide), preferably greater than 100 bar, in order to activate the recirculation of fluid passing through the loop, while contributing to making the treatment flow dynamic. In the reactor, the treatment flow may correspond to a flow which substantially follows a model of axial dispersion in the reactor between the inlet and the outlet (case of piston flow). The open state allows the inlet and outlet of the reactor to be in fluid communication with a respective end of the two ends of the loop.

The opening/closing assembly may also be configured into at least one other state with the control valve open, for example a state (second state) compatible with activation of a separation stage without depressurization or a state (third state) compatible with a depressurization of the reactor and loop.

In this other state, some of the valves remain open and it is possible to discharge co-solvent and the residues which are liquid and/or solid, formed during treatment of the tissue and remaining within the internal volume and in the loop. For this type of state, the loop may be modified locally and typically may have/be connected to a bypass where an outlet/discharge for the co-solvent and residues is formed (without the possibility of returning to the loop, the loop thus being ready for another chemical treatment thereafter).

Optionally, some valves of the opening/closing assembly are configured in a closed state (for example with a backpressure regulator that is blocked off/closed or not fed, by shutting a valve provided at the outlet of the reactor), in order to allow the pressure to increase within the internal volume of the reactor, under the effect of pumping by the pump, the closed state being maintained during a preliminary pressure-increase phase.

The separation stage is for example connected to bypass a section of the loop and is accessible by a given setting of valve(s) of the opening/closing assembly, without being accessible via the loop in a recirculation phase (due to a different setting of the valve(s) concerned), during the phases of tissue treatment with the flow of additive-containing carbon dioxide.

In some options, the system has a co-solvent pump, for example shared for each type of additive, which may be activated during a phase of looping circulation of carbon dioxide in and outside of the reactor, while transitioning to the supercritical state before flowing into the internal volume of the reactor.

The geometry of the reactor is possibly in the form of a column. The height/diameter ratio may be on the order of 4 to 5 for the reactor in this option. A vertically elongate shape with a reduced cross-section or a cross-section matching the transverse size of the area of contact corresponding to the position of the tissue(s), makes it possible to ensure that the supercritical carbon dioxide mainly passes through the material, minimizing peripheral flows which bypass the tissue to be treated.

The ratio between the capacity of the loop and the internal volume of the reactor may be less than 2, for example around 1.

According to one particular feature, the loop is provided with a separation stage (such as a gravimetric or cyclone separator), for separating out the co-solvent and residues discharged from the reactor by circulating in the loop. Such a separation stage may allow separating out residues discharged from the reactor by circulating in the loop. The separation stage is for example arranged in the loop downstream of the pressure-regulating valve of the reactor. A filter may also be provided in the loop.

According to one particular feature, the loop is provided with a filtration stage (filter) which typically allows preventing extracted solid particles from clogging the control valve (which is for example a needle valve sensitive to obstruction, with a very small opening to allow maintaining the pressure upstream). The filter, or similar separating means, thus constitutes a protective means for the control valve. In some embodiments, the filter is a component which may be dismantled and cleaned (at least partially reusable) at the end of a complete cycle. The filter may be arranged at the outlet of the reactor and the pressure-regulating means is for example interposed, in the loop, between the filter and two parallel sections of the loop, one of these sections including the separation stage.

The filtration threshold of the filter may be set at one or more tens of micrometers, in order to trap impurities. This is, for example, a purely mechanical filtration.

The system may comprise:
a source of liquid carbon dioxide connected to the loop, upstream of the carbon dioxide circulation pump;
a source of chemical additive for purification and/or decontamination, connected via a fluidic communication coupling with a section of the loop that extends between the carbon dioxide circulation pump and the reactor inlet;
a discharge line, in fluidic connection to the reactor when recirculation is activated, in order to discharge at least some of the carbon dioxide and the chemical additive for decontamination, without depressurizing the reactor.

Optionally, the system has one or more of the following arrangements:
the discharge line includes a backpressure regulator/regulating valve and forms a section of the loop.
the backpressure regulator is configured so that the discharge line circulates CO₂ in the supercritical state between the reactor outlet and the backpressure regulator.
the source of supercritical carbon dioxide is possibly in the form of a bottle with a dip tube, for delivering the liquid-state carbon dioxide into the loop.
the filter includes a filter medium. An area upstream of the filter medium, delimited in a filtration chamber, may have a tank or bottom for collecting residues. It is understood that the filter is crossed by the treatment flow (the flow having already circulated in the internal volume of the reactor) during an additive injection phase and for example during a phase of closed-loop circulation with no supplying of additive.

### BRIEF DESCRIPTION OF DRAWINGS

Other features, details and advantages will become apparent upon reading the detailed description below, and upon analyzing the attached drawings, in which:

FIG. 1 is a diagram of a system for implementing a method for purifying tissues, provided with a reactor, a recirculation loop provided with a fluidic-connection coupling device which allows injecting one or more additives, it being possible to insert each additive during a treatment cycle that uses the recirculation loop associated with the reactor.

FIG. 2 is a flowchart of steps involved in the method for purifying tissues according to one non-limiting exemplary embodiment.

FIG. 3 illustrates a path for supplying liquid product, associated here with several tanks, with a pump which constitutes the co-solvent pump in a treatment method using a fluid in the supercritical state.

### DESCRIPTION OF EMBODIMENTS

Several examples of non-limiting embodiments are detailed below. In the various figures, identical references indicate identical or similar elements.

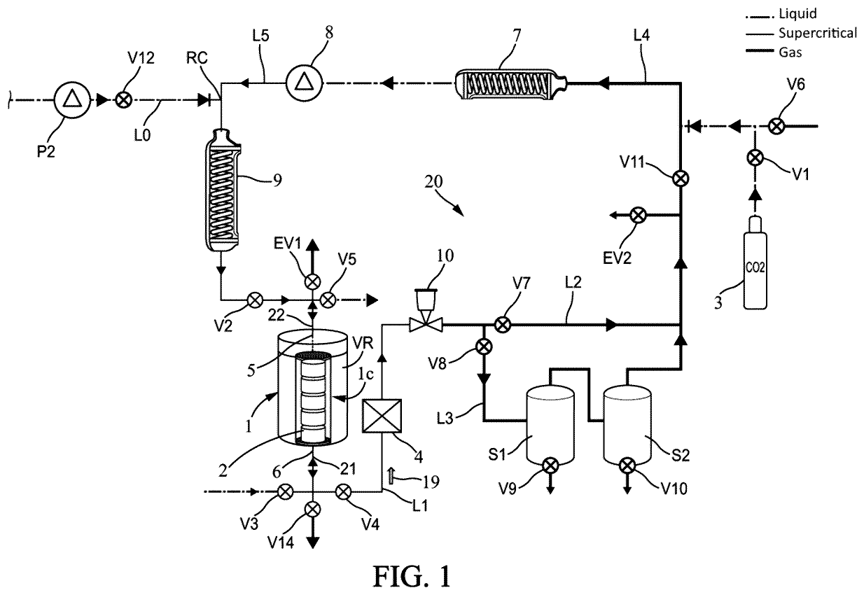

With reference to FIG. 1 which shows a system for implementing the method, a reactor 1 is provided which allows reaching a desired pressurization level. In the system, reactor 1 makes it possible to place the tissue 2 to be treated/purified, within its internal volume VR. Tissue 2 is a biological tissue (of human or animal origin), for example based on a bone matrix, which may be in several pieces or in a single block. In the examples described below, the treatment method aims to purify and/or decontaminate collagen-based tissue matrices. The purging and/or rinsing operations in reactor 1 will be described briefly, where these steps do not specifically relate to the extraction action performed by a solvent (supercritical fluid such as CO₂) nor the combined action carried out by this solvent and a co-solvent.

### Example of a System

Reactor 1 of the system has an inlet 5 and an outlet 6 in order to allow a flow of carbon dioxide CO₂ in the supercritical state to take place through reactor 1. A valve V1 may be associated in a manner that is known per se with a source 3 of carbon dioxide, possibly by forming a fluidic communication coupling with a loop 20 which will be described in detail further below. The pressurization device includes a pump 8, for example a pump for adjusting the flow rate of the carbon dioxide. Pump 8 is capable of compressing the liquid carbon dioxide to a pressure which allows it to transition to the supercritical state. Here, a valve V2 is arranged downstream of pump 8, which can allow reactor 1 to be isolated for loading or unloading.

The system may take the form of a circuit, having a loop 20 passing through a point or zone for inserting carbon dioxide in the liquid state. Such a loop 20 extends for example between a first loop end 21 connected to outlet 6 of reactor 1 and a second loop end 22 connected to inlet 5 of reactor 1, as in the non-limiting case illustrated in FIG. 1. Loop 20 thus associated with reactor 1 makes it possible to form a circuit compatible with closed-loop circulation. More generally, loop 20 may allow the recirculation of fluid coming from reactor 1, from outlet 6 to inlet 5.

As is clearly visible in FIG. 1, a line L5 for supplying the reactor with CO₂ may be formed, on which are successively arranged (in series):
the refrigeration unit 7;
the pressurization device with pump 8, which here forms a high-pressure pump;
a heater 9, for example in the form of a heat exchanger which uses the circulation of a heat transfer fluid, or a resistor.

Before its insertion into reactor 1, the carbon dioxide is thus heated by heater 9 so that at the outlet of this heater, the carbon dioxide is in the supercritical state. In some embodiment options, the supercritical state may be obtained upstream of this heater outlet 9, for example at the outlet of pump 8. The heating may be carried out to obtain a temperature between 31° and 60° C.

Reactor 1 is capable of being closed off and pressurized to maintain the supercritical state of the flow of carbon dioxide. However, it is permitted to let a flow escape, typically through outlet 6 (here with an open valve V4), which circulates in a loop 20 which then ensures its return to reactor 1. The carbon dioxide flow may be gaseous, for example at a temperature of approximately 50° C., in a section L2 or L3 of loop 20, the pressure being lower than what is prevailing in reactor 1, for example around 50 bar. Cooling may be provided in loop 20, in an intermediate section L4, to allow liquefaction of the carbon dioxide in order to facilitate its circulation and its compression by pump 8.

During this type of (looping) circulation to pass through reactor 1 and loop 20 and return to inlet 5 of reactor 1, a bypass or section L3 on which optional separator means S1, S2 are arranged to separate residues from the gaseous CO₂ (for example by performing a liquid-gas and/or solid-gas separation) is not accessible. Inlet valve V8 in this gaseous CO₂ purification branch is kept closed, while valve V7, associated with line L2 which does not pass through separator means S1, S2, is open. Separator means S1, S2 may be of the cyclonic or gravimetric type.

Loop 20 may then successively include, starting from outlet 6 of reactor 1:
a discharge line L1 as the first section of loop 20, which forms an end 21 of loop 20 (here connected to outlet 6 of reactor 1) and joins a valve 10 forming a backpressure regulator;
a second section L2 for the circulation of gaseous CO₂ which leads to the open recirculation valve V11 and allows the flow of CO₂ to continue (as can be seen in FIG. 1, a vent EV2 may be provided just upstream of valve V11);
a third section L3 constituting a bypass to line L2, allowing the gaseous CO₂ to circulate in a separation stage S1, S2 before joining recirculation valve V11;
a fourth section L4 for the circulation of CO₂, which extends from valve V11 to pump 8, allowing the circulating carbon dioxide to cool and liquefy; and
the feed line L5, a fifth section of loop 20, with a circulation of CO₂ in the liquid then supercritical state, forming a second end 22 of loop 20 for connecting to inlet 5 of reactor 1.

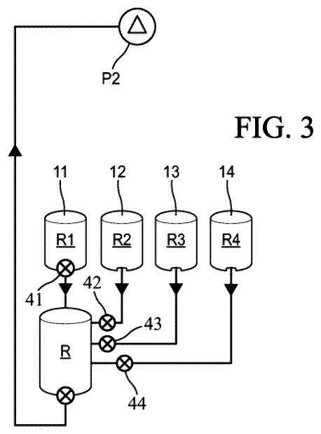

More generally, loop 20 may have many forms. Although loop 20 is shown in FIGS. 1 and 3 as linear, such a loop 20 may have different branches in parallel, for example upstream of pump 8 and/or downstream of pump 8. A reactor inlet 5 corresponding to one end 22 of loop 20 has been described. However, provision may also be made to distribute the flow by using several inlets to reactor 1.

In some variants, all or part of loop 20 may also be used to contribute to the treatment of tissues 2 distributed/allocated to two reactors in parallel. In this case, it is sufficient for example to divide the respective ends 21 and 22 of loop 20, so as to correspond to the corresponding outlets 6 and inlets 5 of reactors 1.

As illustrated in the non-limiting example of FIG. 1, reactor 1 of the system may be interposed between pump 8 and the valve forming a backpressure regulator 10. Pump 8 may be designed and arranged to:
transition the carbon dioxide from the liquid state to the supercritical state, upstream of inlet 5 of reactor 1 in the direction of circulation (from pump 8 to inlet 5);
in a mode with recirculation (as illustrated for example in FIG. 1) which uses an additive injected as a co-solvent downstream of pump 8, carry out the pressurization in the presence of co-solvent recirculated with carbon dioxide as solvent.

The system has a fluidic connection device RC for connecting with loop 20, which is for example associated with additive insertion means V12. In the non-limiting embodiment of FIG. 1, such a device RC is illustrated upstream of heater 9 but an arrangement upstream of pump 8 or closer to reactor 1 (close to inlet 5, for example downstream of heater 9) is of course possible.

Means V12, which include for example an insertion valve coupled where appropriate to several respective tanks R1, R2, R3, R4, may deliver a liquid-state solution directly into a CO₂ flow in the supercritical state, meaning into the flow of line L5 subjected to a high pressure, for example between 100 and 200 bar. Alternatively, the solution is delivered further upstream, into a CO₂ flow that is still liquid. In the non-limiting case of FIG. 1, means V12 may allow adding at least one chemical additive 11, 12, 13 for purification/decontamination of the tissue, in a section of loop 20 located downstream of pump 8 and upstream of inlet 5, such that connection device RC forms a mixer. Thus, a mixed/heterogeneous flow combining carbon dioxide in the supercritical state as a treatment solvent and the additive as a treatment co-solvent may be conveyed together into the internal volume VR of reactor 1 in order to reach tissue 2. Means V12 may be configured so that the liquid flow rate is significantly lower than the CO₂ flow rate, in section L5 of loop 20 (and more generally in the entire circuit).

Regardless of the point of insertion (chosen just upstream or downstream of pump 8), a pump P2 is provided, referred to in the following as a co-solvent pump, which is part of insertion means V12. Pump P2 is configured with a flow rate which depends on the flow rate of pump 8, in order to ensure dissolution of the co-solvent in the solvent (carbon dioxide in the supercritical state). For example, the ratio of additive flow rate (co-solvent)/solvent flow rate may be between 1/5 and 1/100. In some exemplary embodiments, this ratio is 1/50. At the inlet of reactor 5, a treatment flow which contains the additive may thus be admitted. As described below, the chemically active additive may be inserted when diluted in another liquid, preferably with a possibility of controlling the concentration of the chemical component involved in the tissue treatment for eliminating impurities/residues, in particular a possibility of controlling a ratio between a parameter representative of the mass or total concentration of additive inserted into reactor 1 and the total mass of tissue to be treated.

Reactor 1 is designed and arranged to allow the entry of an additive as a co-solvent, when internal volume VR is already pressurized. To allow activation of loop 20 while maintaining this pressurized state, the system may include an opening/closing assembly provided with valves (V2, V4, 10, V7, V11); such a set of valves may have an opening configuration compatible with a circulation in series between the reactor and the loop. This assembly may allow the recirculation of fluid, in a first open state, the pressure-regulating valve 10 allowing the pressurization of reactor 1 to be kept above a threshold, preferably greater than 100 bar.

In the case illustrated in FIG. 1, many valves or flap/vent systems are shown. Of course, loop 20 may have fewer valves, for example by integrating a different valve system at least in certain parts of the circuit, integrating valves directly in the separator means S1, S2, or other comparable arrangements.

At the end of a treatment cycle in the pressurized reactor 1, the device with valves V7, V8 that is provided downstream of backpressure regulator 10 may be modified/configured differently to use bypass line L3, in order to be able to access separator means S1, S2. More generally, purging steps may use all or part of loop 20 as well as one or more separators S1, S2, which are typically separate from filter 4 provided upstream of valve 10. Here, the co-solvent and the residues of materials carried along by/dissolved in carbon dioxide which have remained in reactor 1 are discharged via the reactor outlet 6, circulated through valve V8 as bypass valve V7 is closed, and collected at separator means S1, S2, here in this example at the outlet of a first separator S1, via a purge valve V9. The facility/system may also comprise a second separator S2, this second separator S2 having a valve V10 for purging organic materials. More than two distinct separator means may be provided, depending on requirements.

The step of recovering residues and co-solvent via the separation stage/means S1, S2 is carried out while the carbon dioxide is circulating in section L3, so that this solvent is recycled in the circuit by means of a valve 11 that joins section L4, valve V7 being closed.

### Steps of the Method

After tissue 2 has been placed within the internal volume VR of reactor 1, possibly arranged on a suitable container or support, tissue 2 may remain in a fixed position and an area of contact 1c is defined, located between inlet 5 and outlet 6. When reactor 1 is arranged vertically, for example by forming a column, tissue 2 may thus be placed at an intermediate height between inlet 5 which is located at an upper end of reactor 1 and outlet 6 formed at a lower end of reactor. A cover including inlet 5 may allow sealing off the internal volume VR delimited by the reactor body. The access ports to reactor 1 may be limited, for example primarily including inlet 5 and outlet 6 and possibly an additional port forming a vent.

In order to purify/decontaminate tissue 2, the method may begin by a first treatment cycle with, firstly, inserting carbon dioxide in the supercritical state into the internal volume VR, for example by configuring the opening/closing assembly to place in communication:
outlet 6 with section L1, as can be seen in FIG. 1 for example;
inlet 5 with section L5.

The carbon dioxide may circulate in a loop while being under pressure in reactor 1, in the supercritical state.

This corresponds to a first circulation, which is for example a circulation without addition. With reference to the example in FIG. 1, valve V4 shown in FIG. 1 may then be opened (reactor drain valve V14 closed), while backpressure regulator 10 allows fluid to circulate towards recirculation valve V11. Sections L4 and L5 are involved, when starting up a cycle, in order to allow the reactor to be filled with CO₂, and are also used in a similar manner during such a first circulation.

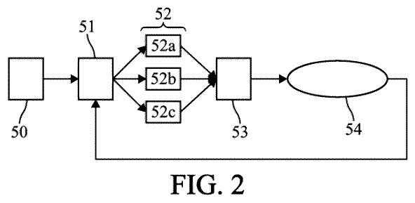

Referring now to FIG. 2, the successive steps of placing 50 the material to be treated in reactor 1, and of supplying/distributing 51 CO₂ may constitute steps of the method which precede step 52 of inserting additive(s) via an appropriate valve V12 connected to supply line L5. The filling of reactor 1 with CO₂ when pressurized, for example at 160 bar, and the filling of loop 20 may correspond to a limited duration, in all cases less than the duration of the treatment steps.

During step 51, pump 8 operates to ensure that reactor 1 is traversed by a dynamic flow of supercritical CO₂. An example flow rate delivered by pump 8 is a continuous flow rate of 5 kg/h under stable temperature and pressure conditions. At the end of step 51 and for step 52a, 52b or 52c of inserting a co-solvent (with additive), pump 8 remains running. While the circulation of carbon dioxide is looping in the circuit (reactor 1+loop 20), and a desired pressure and temperature are reached in reactor 1, for example 160 bar and 40° C., and the pressure/flow rate/temperature parameters are stable, the insertion of at least one co-solvent can begin. The injections carried out in steps 52a, 52b or 52c may be configured with predefined proportioning, taking into account a parameter representative of the total amount of additive injected which is calculated/proportioned according to the total mass of tissue 2 to be treated, present in reactor 1, independently of the capacity of reactor 1 or the location/distribution of tissue 2 in the internal volume VR.

While the carbon dioxide in the supercritical state is dynamically circulating in reactor 1, beginning its cleaning action (in particular delipidation) in the area 1c of contact with tissue 2, insertion means V12, P2 deliver the co-solvent, using for example a co-solvent pump P2 which delivers the co-solvent in the liquid state in a co-solvent supply line LO. A chemical additive 11, 12 or 13 may thus be added for complementary action, for example chemical/enzymatic, carried out jointly with the action of carbon dioxide in the supercritical state, and while following the flow of this solvent.

Once additive 11, 12 or 13 has been injected into reactor 1 as a co-solvent, typically at a comparatively low volume flow rate compared to that of CO₂, a flow different from the flow circulating during the first circulation with CO₂ is recirculated in loop 20: a second type of circulation is obtained corresponding to a dynamic treatment flow with additive. The opening of co-solvent insertion valve V12 and the starting of pump P2 are carried out without interrupting the circulation, so as to inject a volume of liquid into the pressurized CO₂, therefore typically here without stopping pump 8. In some options, the reheating of the heterogeneous flow (CO₂ in the supercritical state or close to this state, and additive) by heater 9 can make it possible to maintain stable parameters in reactor 1, without the temperature, pressure, or flow rate varying significantly at inlet 5.

The system allows for proportioning the amount of additive, by controlling:
on the one hand, the volume flow rate of the co-solvent (which may be a solution with the chemical agent diluted in the liquid solution), this flow rate able to remain substantially constant by adjusting pump P2;
and on the other hand, the injection duration.

In some variants, the amount of additive may also be predetermined without relying on injection at a constant volume flow rate, for example by using a gradient or a controlled variation in the flow rate, or by alternating injection phases with pauses, while controlling/setting in advance the total amount injected per sequence.

The system may allow implementing a treatment method with a sequence of treatment phases using a particular additive. Here, the same point of insertion formed on section L5 of loop 20 may be used by using the same additive insertion device or means (P2, V12). For example, FIG. 1 shows the use of a connection coupling RC, which may optionally be comprised in the co-solvent inlet valve V12. The liquid additive may be inserted into a section of the loop where the carbon dioxide is either liquid or supercritical. For example, this additive may be inserted before reactor inlet 5, optionally upstream of pump 8, starting at the condenser 7 outlet.

With reference to FIG. 3, pump P2 may receive liquid conveyed from a co-solvent tank R which may be filled with different additives 11, 12 or 13, mixed when appropriate with a substance 14 such as water or other solvent (aqueous or not). The concept of co-solvent can thus designate a solution injected into the flow of CO₂ (supercritical or possibly still liquid) and which will be placed in contact with the material to be treated (tissue 2). The co-solvent may contain at least one active ingredient and an associated diluent.

For example, a first tank R1 is provided for a first additive 11, for example hydrogen peroxide. Other tanks R2 and R3 may be used to store a second additive, for example an acid such as PAA, and a third additive or component, for example ethanol. Respective valves 41, 42, 43, 44, associated with each of these tanks R1, R2, R3, R4 are illustrated here. By opening valves 41 and 44, simultaneously or not, in order to fill co-solvent supply tank R, with control of the flow rate, it is possible for example to produce a 35% hydrogen peroxide solution (or other desired percentage). Similarly, using valves 42 and 43, it is possible to produce a solution of peracetic acid diluted in ethanol at a defined percentage, for example 18-20% peracetic acid (or other desired percentage depending on requirements). We have cited the case of H₂O₂ and peracetic acid as active ingredients. Of course, other molecules or substances may be used, to the extent that such a substance, combined with supercritical carbon dioxide, has a purifying or decontaminating action on the material to be treated.

Of course, in other options, tank R may be eliminated and different co-solvent supply lines can be used; where appropriate, by making use of additive insertion means arranged differently, for example in parallel with each other up to the point where they join loop 20, downstream of pump 8.

The treatment of matrix/tissue 2 in reactor 1 is carried out, at each treatment cycle, by a flow of supercritical carbon dioxide, supplemented by the insertion (for example during the circulation of CO₂ in reactor 1 and loop 20) of at least one chemical additive injected by insertion means P2, V12 in the form of a liquid solution while reactor 1 is already pressurized. The combination of supercritical CO₂ as a solvent and the chemical additive as a co-solvent may allow increasing the cleaning/disinfecting action on the treated tissue. At outlet 6 of pressurized reactor 1, the flow circulates in end 21 of loop 20 then joins the rest of the loop via backpressure regulator 10. The circulation of a first additive, progressively injected then recirculated with the CO₂, is part of a first treatment cycle. Several cycles, each with an additive, may follow one after the other, separated by separating out the co-solvent and the residues at constant pressure or a depressurization and a purge, as detailed a bit further below.

In some options, the valve forming backpressure regulator 10 is managed and regulates the upstream pressure by allowing a trickle of fluid to pass through, here fluid based on carbon dioxide. As a non-limiting example, between valve 10 and pump 8, the pressure prevailing in loop 20 (in sections L2, L3 and L4) may be approximately 50 bar.

### Example of Operating Parameters

Here, carbon dioxide CO₂ is pumped in liquid form by pump 8. This liquid is preheated upstream of extraction reactor 1 in order to be inserted into the reactor in the supercritical state.

Recall that fluids in the supercritical state can be defined as gases placed under temperature and pressure conditions such that their properties are intermediate between those of gases and those of liquids. They are also called “dense gases” or “gas-expanded liquids”. For a given chemical body, the precise point in the temperature-pressure diagram at which the two phases, liquid and vapor, form only one phase is called the critical point. Beyond this critical temperature (Tc) and this critical pressure (Pc), the fluid is in the so-called “supercritical” state.

The system uses carbon dioxide in the supercritical state, at least in a portion of the loop corresponding to section L5 and in the internal volume VR of the pressurized reactor. By passing through reactor 1, carbon dioxide in the supercritical state makes it possible to solubilize a large portion of the organic matter, essentially lipidic, of tissue 2. In particular, it can dissolve the fats of the medullary tissue contained in bone tissue.

To obtain this action, the implementation conditions may vary. To illustrate the concept, the following conditions are given as a non-limiting example:
the pressures prevailing in reactor 1 range from 100 to 200 bar, so a pressure of 160 bar may be chosen. Such pressure conditions are compatible with temperatures that remain well below 90 or 100° C., for example temperatures between 35 and 50° C.: a temperature of 40° C. may optionally be chosen.
for a given amount of material to be treated (total mass of tissue 2 in reactor 1 in the non-limiting example of FIG. 1), a total volume of co-solvent to be injected may be determined by the operator according to the nature of the co-solvent, its concentration, and the desired effect: a usable parameter is the ratio of co-solvent/mass of material (tissue 2), expressed in ml/g. This is called the first ratio in all that follows. A first ratio of 0.25 ml/g is chosen in some of the experimental cases that follow but, of course, a completely different ratio could be suitable (in particular, this ratio necessarily changes according to whether the active ingredient is more or less diluted in the co-solvent solution).
it is possible to adjust the flow rate of co-solvent pump P2 according to the flow rate of supercritical CO₂ pump 8, so as to ensure dissolution of the co-solvent in the solvent. The ratio of co-solvent flow rate/supercritical CO₂ flow rate, hereinafter called the second ratio, may vary depending on which co-solvent is conveyed via insertion means P2, V12. With the solutions envisaged, the second ratio is for example between 1/5 and 1/100: a ratio of 1/50 may be chosen.

The facility or system, in particular reactor(s) 1 and the type of associated loop 20, may vary in its structure and capacity, which may depend on the total mass of tissue 2 arranged in a given internal volume VR. Optionally, volume VR is greater than the volume of loop 20 which completes the circuit (here, the volume of the loop does not include the separating means S1, S2 which are not used in the closed-circuit recirculation). Depending on the volumes of reactor 1 and loop 20, a flow rate may be applied for pump 8 that is adapted to the desired effect on tissue 2.

At each step in the treatment, secondary parameters (variables) of the method may be deduced from primary parameters as indicated above.

Thus, with a known flow rate of pump 8 (flow rate of supercritical CO₂), the co-solvent flow rate may be deduced from the second ratio. Similarly, the volume of co-solvent inserted may depend on the amount of material to be treated, based on the first ratio mentioned above.

The injection time may be calculated based on the co-solvent flow rate and the volume (total volume) to be injected via insertion means P2, V12. The time taken to traverse reactor 1 is calculated based on the flow rate of the supercritical carbon dioxide and internal volume VR of reactor 1. Furthermore, the initial contact duration of the co-solvent with the material/tissue 2 (corresponding to the duration of the first passage of co-solvent over the material) is typically equal to adding the injection time and the time taken to traverse reactor 1.

Once the planned volume of co-solvent has been injected, the co-solvent (with the corresponding additive 11, 12 or 13) circulates in the circuit until it completes a full cycle through the system. The time of a cycle is defined by the flow rate of pump 8 and the sum of volume VR of reactor 1 and the volume of the rest of the circuit (here loop 20). If there is a need to obtain a longer contact duration between the co-solvent and the material, it is sufficient to re-cycle the mixture (supercritical solvent/co-solvent). Optionally, a certain number N of round trips may be configured, once all the co-solvent has been inserted into the circuit by reaching section L5.

### Practical Experimental Example

In this example, a total mass of 320 g of bone tissue is placed in reactor 1 and the envisaged method provides for several types of treatments, one after the other:
first by hydrogen peroxide, for example with this active ingredient being added in a 35% liquid solution (possibly an undiluted commercial solution),
then by PAA (peracetic acid) diluted 2.7 times in 99% ethanol, i.e. a PAA concentration of 18% in the co-solvent solution,
then by 99% ethanol.

In this non-limiting example, the volume of each co-solvent to be applied is predetermined to be 0.25 ml of co-solvent to treat 1 g of material (for example 1 g of bone). The system may have the following features:
a circuit with a volume of approximately 2 L, of which 1 L corresponds to the internal volume VR of reactor 1.
pump 8 is configured to deliver a flow rate of 5 kg/h or approximately 104 ml/min (for a density of supercritical CO₂ of approximately 0.8 at 160 bar). The second ratio for the flow rate (volume flow rate) of pumps P2 and 8 is 1/50.

The treatment steps are carried out at 160 bar and 40° C. in reactor 1. The values deduced are as follows:
The co-solvent flow rate is 2.08 ml/min (104/50).
The volume of co-solvent to be injected is 80 ml (0.25*320).
The injection time is 38 minutes 27 seconds (80/2.08); this time can obviously be rounded to 38 or 39 minutes.
The time taken to traverse the reactor is 9 minutes 37 seconds (1000/104).
The time of one cycle within the circuit for a complete round trip is approximately 20minutes (2000/104).

The experimental example is carried out with recirculation for 100 minutes, or 5 treatment cycles, to obtain a total contact duration (in area of contact 1c) of 138 min, using a given additive/active ingredient at each step.

The complete sequence of phases of the treatment method, here based on the set values (but which are not limiting), corresponds to the following:
a. Inserting the material to be treated (tissue 2) into reactor 1
b. Continuous flow of supercritical carbon dioxide at 5 kg/h at 160 bar and 40° C., using pump 8
c. Inserting 35% hydrogen peroxide for 38 minutes (80 ml), upstream of reactor 1 in section L5, using insertion means P2, V12 (including pump P2), this active substance forming a first additive 11
d. Maintaining a (closed) recirculation loop for 100 minutes (5 round trips in the closed circuit)
e. Purging reactor 1 and the rest of the circuit then rinsing with purified water to eliminate the residues of material extracted from tissue 2
f. Recharging with carbon dioxide then restarting the flow of supercritical carbon dioxide at 5 kg/h at 160 bar and 40° C.
g. Inserting 18% PAA for 38 minutes (80 ml) upstream of reactor 1, using insertion means P2, V12 (including pump P2), this active substance forming a second additive 12
h. Maintaining the recirculation for 100 minutes (5 round trips)
i. Purging, for example as a result of activating separation stage S1, S2 in order to retrieve and purge the PAA for 60 minutes
j. Inserting 99% ethanol for 38 minutes (80 ml) upstream of reactor 1, using insertion means P2, V12 (including pump P2), this active substance forming a third additive 13
k. Maintaining a (closed) recirculation loop for 50 minutes (2.5 round trips)
l. Purging, for example as a result of activating of separation stage S1, S2 in order to retrieve and purge the ethanol for 60 minutes
m. Inserting 99% ethanol for 38 minutes (80 ml) upstream of reactor 1, using insertion means P2, V12 (including pump P2)
n. Maintaining a (closed) recirculation loop for 50 minutes (2.5 round trips)
o. Stopping pump 8 and purging reactor 1 and the rest of the circuit (loop 20) in order to eliminate the ethanol
p. Removing the cleaned tissue (treated material) from reactor 1.

Of course, the main treatment steps (a-e/or f-i/, j-l and/or m-p/) may be adjusted as needed, where appropriate by substituting one active ingredient for another (therefore changing the co-solvent). With reference to FIG. 2, after step 50 of placing tissue 2 in reactor 1 (see phase a/indicated above), once reactor 1 is closed and provided with sealed connections in communication with ends 21, 22 of loop 20, and after step 51 of filling with and dynamically circulating supercritical CO₂ (which may correspond to phase b/, possibly repeated in f/, j/or m/), it is understood that step 52 of inserting additive is carried out in a progressive and controlled manner to achieve a relatively low ratio between the total volume of injected co-solvent and the mass of tissue 2 to be treated in reactor 1.

The experimental example detailed above corresponds to one particular case. Optionally, the step of ethanol treatment or other last treatment/rinse may include fewer sub-steps or does not require two ethanol rinse cycles, this number of treatments possibly being higher or lower. In other examples, in order to treat more tissue 2 (greater mass), regardless of whether the volume of reactor 1 is different (larger), a similar injected volume of co-solvent may be used but having a higher concentration of active substance. Multiple variants are applicable concerning the exact mode of injection and the duration of each recirculation, while predicting in advance the amount of active substance (co-solvent) based on the mass of tissue 2 to be treated.

Limiting this total amount may prove beneficial to preserving the collagen tissue matrix, and to preserving the mechanical properties of the material to be treated. Phases c/and d/respectively correspond to step 52a of injecting the first additive and to step 53 of recirculating a (mixed) treatment flow. Phases g/and h/respectively correspond to step 52b of injecting a second additive and to repeating step 53 of recirculating the (mixed) treatment flow obtained. Similarly, step 52c corresponds to the step of injecting a third chemical agent, for example ethanol. Arrow 19 after outlet 6 of reactor 1 indicates the residues and illustrates the fact that residues from the treatment are carried into loop 20, with the possibility of extracting/separating some of them so as not to return these residues to reactor 1, with the knowledge that the treatment options may require one or more complete round trips during recirculation step 53.

Discharge/cleaning step 54, corresponding to the end of a cycle, provides for the depressurization of reactor 1 with the use of vent EV1, which may include a valve. Such a vent EV1 may here be an upper vent on a column-type reactor, possibly formed at inlet 5. Pump 8 is stopped in order to slowly depressurize (approximately 10 bar/min down to a first threshold, for example equal to 100 bar, then a slower decrease, here 4 bar/min down to 73 bar or a comparable second threshold, and 2 bar/min until atmospheric pressure is obtained) by opening vent EV1. Step 54 may include purge and/or rinse sub-steps. For example, reactor 1 may be drained by opening its valve V14 coupled to outlet 6 (here a lower drain valve) and by closing the valve forming vent EV1 and by opening the control valve 10 forming a backpressure regulator. This is followed by purging the co-solvent residues, carried out for example with compressed air for a defined duration which is less than the duration of a treatment step with looping circulation, for example on the order of 10 to 60 minutes. After 5 minutes of purging, recirculation valve V11 may be closed.

It should be noted, in some options which allow cleaning the discharged carbon dioxide, that line L2 is not used during step 54, valve V8 being open (with valve V7 closed). Thus, separating means S1, S2 separate out the residues discharged during step 54 via line/section L3.

Although not illustrated in FIG. 2, step 54 with purging may include or be followed by a rinsing step using purified water, for example when the chemical additive used is H₂O₂ or a comparable oxidizing and disinfecting active substance, typically diluted in water.

As an example of rinsing with purified water, the following sequence of operations may be provided, starting with a purged reactor 1 (having undergone the basic purge phases of step 54):
performing a first injection of purified water (if applicable from tank R4 or another available tank), which reaches area of contact 1c via a reactor inlet valve V2, for example by means of a pneumatic or electric pump.
opening drain valve V14 (lower drain of reactor 1 here in the example of FIG. 1), with valve V4 (reactor outlet and forming an access valve to loop 20) being closed, which makes it possible to avoid interfering with loop 20.
once the water drained (via outlet 6) is clean, closing valve V14.
closing valve V2 and opening purified water inlet valve V3 connected to outlet 6, here corresponding to the lower port of reactor 1.
opening valve V5 associated with inlet 5 of reactor 1, so as to allow an upper purge, i.e. discharging the rinse water.
optionally, measuring the peroxide level at the outlet end of valve V5 (which here is an upper purge valve), then diluting the solution to 1/100th before measuring the remaining level of peroxide. It is verified that the level is below a threshold, for example 2 mg/l, then the injection of water via outlet 6 is stopped.
in order to purge the water from reactor 1, opening inlet valve V2 of reactor 1, opening valve V14, and injecting compressed air. Valve V5 is closed as soon as there is no more water exiting through valve V5.
purging residues, in particular co-solvent residues, with compressed air for 60 minutes, by injection with valve V2 kept open, while leaving valve V4 open so that the purge applies to loop 20. After 5 min, closing recirculation valve V11.
stopping the supply of compressed air.

The compressed air may come from a point or zone of insertion that is separated from reactor 1 by at least one loop section provided with one or more valves (valve V2 on the inlet 5 side; valve V11 and/or valve V4 on the outlet 6 side). For example, this point of insertion may correspond to a connection upstream of refrigeration unit 7, with a compressed air supply valve V6. Compressed air may optionally be injected through a coupling that is shared with the supply of liquid CO₂, as illustrated in FIG. 1.

Of course, the rinsing may vary in its implementation, for example by modifying the system, by modifying the duration or certain control parameters enabling the transition between rinsing with purified water and purging with compressed air. Each purge constitutes a way of stopping the action of the co-solvent and at the same time eliminating the residues extracted from the material (tissue 2) via the combined action of the solvent (supercritical fluid) and the co-solvent (chemical additive).

After step 54, supplemented or not by rinsing with purified water, the system may be prepared once again for another step of treatment by dynamic flow with additive-containing solvent. For this, loop 20 and reactor 1 (therefore the circuit) may be filled with CO₂, the recirculation mode may be activated, and pump 8 is started up to circulate CO₂ in the supercritical state in reactor 1, before proceeding with inserting a new additive.

With reference to FIG. 1, the carbon dioxide collected via one end 21 of loop 20 successively transitions to the gaseous state, downstream of a control valve called a backpressure regulator 10, then into a liquid state in order to continue to circulate in loop 20. Filter 4 allows the retention of residues, for example solid residues. Here, this filtration is performed on line L1 where the carbon dioxide is circulating in a supercritical state. The recirculation downstream of valve V11 allows pump 8 to bring a treatment flow without solid residues back into reactor 1, with the advantages of a solvent fluid in the supercritical state, this flow forming a dynamic flow.

In order to facilitate contact, an additional stirring action may be provided, for example by using an ultrasonic device, if necessary with a movable bar. Such a device may allow detaching the impurities from the material and facilitating their removal, the circulation in reactor 1 towards outlet 6 and loop 20 allowing such impurities to leave the area of contact/treatment 1c. In alternative embodiments, all or part of loop 20 may be formed in or on reactor 1. Where appropriate, an internal partition provided in the reactor may allow separating out some of the residues in a sub-region of the internal volume VR, interposed (in the direction in which the carbon dioxide is circulating) between the area of contact 1c and outlet 6. More generally, it is understood that the pressurization of reactor 1 is compatible with a dynamic effect, which allows the discharge of residues (via a discharge line, here corresponding to section L1 in fluidic connection with reactor 1 when recirculation is activated) towards a second region that is offset/separate from area of contact 1c. By discharging at least some of the carbon dioxide and the chemical additive 11, 12 or 13 for purification and/or decontamination, without depressurizing reactor 1, the discharge line or section L1 (including backpressure regulator 10) contributes to limiting/reducing the concentration of additive in area of contact 1c, while also reducing the presence of residues in area 1c.

One option with a loop 20, for example external to reactor 1, allows ensuring that co-solvent will be removed from area of contact 1c, which thus reduces the concentration of active substance/additive 11, 12, 13 in area of contact 1c. With an external loop 20, reactor 1 can remain simple in design.

At the end of the method, tissue 2 placed in reactor 1 can be recovered after opening reactor 1. A drying phase may then be carried out, for example in a ventilated oven, at a temperature between 30 and 50° C., for example at 40° C. The drying time may be for example between 6 and 12 hours. The duration may be adapted according to the efficiency of the drying device used.

More generally, the duration of numerous steps in the method may vary, depending on the weight of tissue to be treated and the flow rate of supercritical carbon dioxide inserted into reactor 1. Treatment steps 51, 52 of the method may last just long enough for a mass of carbon dioxide and an amount of additive 11, 12, 13 passing through tissue 2 (its mass having been determined) to have been able to react in area of contact 1c. With the use of a loop 20, it is possible to cut off the CO₂ supply fairly quickly, from bottle 3 or similar source, well in advance of the end of the treatment, when the mass of carbon dioxide used is sufficient to fill the volume of the circuit.

The treatment method, with dynamic circulation of supercritical CO₂, in which the additive is added in the form of a liquid co-solvent, allows effective cleaning without degradation, due to a concentration of active product which remains moderate, in particular which remains low compared to more conventional methods using simple soaking and impregnation of the tissue in a chemical agent.

In some examples, the number of co-solvents may be greater than two or three. One particular case has been described with three co-solvents used, in the following order: hydrogen peroxide, PAA, and ethanol, as an additive in the loop where carbon dioxide is circulating in the supercritical state. However, in some variants, a short preliminary treatment may be used, for example shorter in duration, during which the tissue is in the presence of an active substance.

For example, an additive diluted in water, such as hydrogen peroxide, may optionally be inserted at an earlier stage, for example in the form of a first dose already present in the reactor (before any looping circulation if there is such) or injected at the same time as the prefilling with CO₂ in order to achieve the desired pressurization state. Also in this case, contact with the active substance, hydrogen peroxide for example, may be obtained with a low concentration. In some embodiments, the first dose may be taken into account for then calculating the additional supply of additive (hydrogen peroxide or similar) inserted as a co-solvent, preferably progressively. The first dose may represent about 1 or 10% only, or in any case a minority fraction compared to the total amount of additive injected (first dose<progressively injected second dose).

Loop 20 offers the advantage of being able to optimize the effectiveness of the treatment, not necessarily by increasing the number of reagent molecules present in reactor 1 at a given time, but by creating a dynamic flow and for example increasing the frequency at which these molecules pass through a reactive zone (area of contact 1c), by returning the molecules (extracted from reactor 1 by loop 20 via an outlet 6) to inlet 5 of reactor 1 after circulating in a flow directed towards/passing through tissue 2 to be treated.

The total amount of material is put to better use, with improved efficiency, and may be reduced, if necessary by slightly increasing the treatment duration in order to increase the number of pass-throughs and therefore the contact between an active component and the tissue to be cleaned.

The circulation may take place in a reactor 1 having a general column shape, in order to create agitation within the internal volume VR, in a piston flow in the reactor (similarly to the piston of a syringe). The combination of a filter 4 and a separation stage contributes to cleansing under dynamic treatment conditions. Filter 4 allows purification of the flow leaving reactor 1 by separating out solid particles via a purely mechanical separation (without chemical treatment) and with a sub-millimeter filtration threshold, for example greater than or equal to 10 μm. It is understood that the active substances can pass through such a filter 4: there is therefore no reduction in the co-solvent concentration (the circulating additive) due to this separation stage.

One or more separation stages may be provided for recovering the co-solvent or active product, at the end of a treatment phase/step 54. A bypass valve may allow access to the one or more separation stages, only at the end of a treatment cycle or phase.

### Non-Limiting Experimental Practical Case

In one experiment, an animal bone matrix weighing approximately 320 or 330 grams was cleaned for a cycle duration of around 100 minutes for each co-solvent. The purging steps were the same between each cleaning cycle, with the action of a chemically active agent. A satisfactory cleaning quality corresponds to the disappearance of sufficient impurities while retaining conventional properties compatible with implantation (here MC3T3 cell adhesion test on bone matrix, tested after 48 hours, and implantation test on a rat critical-size defect model).

In this example, approximately 320 g of bone tissue is treated with undiluted 35% hydrogen peroxide and PAA (peracetic acid) diluted 2.7 times in 99% ethanol, i.e. a PAA concentration of 18% in the co-solvent solution, then with 99% ethanol. Reactor 1 may have a capacity of approximately 1 liter. More generally, it is understood that the capacity of reactor 1 may vary depending on the amount of material/tissue to be treated.

Bone tissue 2 may be distributed in multiple blocks within the zone/area 1c of contact with the gas flow circulating in reactor 1, for example staggered along the heightwise direction or the main direction of circulation and/or arranged next to each other transversely relative to the main flow direction. Alternatively, tissue 2 may be presented as one block (a non-limiting example may be an entire femur). The method may also be applied to several tendons.

In this treatment, the volume of each co-solvent (corresponding to an additive 11, 12, 13) to be applied is predetermined to be 0.25 ml of co-solvent to treat 1 g of bone. This thus represents 80 ml of added volume, which thus has similarities with the experimental conditions set forth above. The concentrations of co-solvents are measured in ml of active substance per gram of material to be treated, reduced in particular by having them circulate in a flow of supercritical carbon dioxide and by using a circulation loop 20.

The method shows effectiveness in purification/decontamination despite the savings/reduction in active agent(s), which is applicable to any type of bone matrix or tissue of human or animal origin to be treated and with a different number of cycles and/or a choice (quantitative or qualitative) of different cleaning agents.

A comparison concerning bone generation was established for two respective types of tissue blocks, each consisting of a unit of porcine cancellous bone. The tests were carried out on rat critical-size defects for the two types of compared units in order to regenerate a critical size defect. The blocks of the first type were treated (Test A) according to a method as described above, with three successive additives inserted in three treatment cycles with the additive-containing carbon dioxide. The blocks of the second type were treated (Test B) using a method that also provides for three additives, each with a treatment of comparable duration with a chemical action obtained in a different form, namely: after delipidation with supercritical CO₂ under the same conditions, the blocks were successively soaked in a 35% hydrogen peroxide solution, a 1 mol/l sodium hydroxide solution, and a 99% ethanol solution.

Analysis of the results uses a parameter representative of the amount of bone generated in the region of interest, measured by micro-computed tomography. Here, this parameter is the parameter BV/TV (bone volume over total volume), expressed as a percentage.

The results are as follows:

| % BV/TV | 0 days | 30 days | 60 days |
|---|---|---|---|
| Number of | 12 | 12 | 8 |
| defects | | | |
| Test A | 15.8 | 22.3 | 25.9 |
| Test B | 16.7 | 16.5 | 18.3 |

Faster growth in the percentage of coverage by bone material is observed with the units of Test A, obtained according to the method. Thus, it is found that the action obtained with the method is more effective for bone regeneration than for the case of a bone chemically treated with a method (Test B) involving soaking in a liquid phase. In addition, the action of the treatment method (Test A) makes the material regenerate bone faster. Concerning the results, statistically significant differences are obtained:
for the increase in bone regeneration with Test A, with p<0.05 between D0 and D30 and p<0.001 between DO and D60;
for the comparison of bone regeneration between Test A and Test B, with p<0.05 at D60.

This disclosure is not limited to the embodiments described above solely as an example, but encompasses all variants conceivable to a person skilled in the art within the framework of the protection sought.

For example, although one reaction chamber within the volume VR has been illustrated in FIG. 1, an arrangement with several chambers each housing at least one tissue 2 may be provided, using one or more reactors 1. In addition, controlled flow pulses, for example during step 51 and/or one of steps 52a, 52b, 52c, may be envisaged.

Also, it is clear that the number of chemical additive(s) may vary depending on the treatment sought.

1-13. (canceled)

14. A method for treating a tissue of human or animal origin by extracting organic matter residues in order to purify and/or decontaminate said tissue, the method using carbon dioxide in supercritical state and a reactor provided with an inlet and an outlet, the method comprising:
arranging the tissue within an internal volume of the reactor;
pressurizing the reactor, by inserting carbon dioxide in the supercritical state into the internal volume via the inlet, such that the carbon dioxide in the supercritical state reaches an area of contact with the tissue and exits the reactor via the outlet;
adding at least one chemical additive for purification and/or decontamination into a flow of liquid or supercritical carbon dioxide being circulated so as to reach the area of contact, in order to create a circulation of a treatment flow within the internal volume which combines the carbon dioxide in the supercritical state as a solvent and the at least one chemical additive as a co-solvent; and
while maintaining said pressurization, causing said treatment flow to circulate, outside of the area of contact, in a loop that is in communication with the inlet, thereby creating a recirculation which contributes to making the treatment flow dynamic and to purifying and/or decontaminating the tissue,
wherein the carbon dioxide circulating in a loop successively transitions to a gaseous state, downstream of a pressure-regulating valve, then to a liquid state by being cooled.

15. The method according to claim 14, wherein the carbon dioxide is pumped by a pump provided in the loop, the loop extending outside the reactor, between the outlet and the inlet of the reactor,
and wherein the at least one chemical additive is inserted into the loop upstream or downstream of a heat exchanger which heats a flow of carbon dioxide coming from the pump provided in the loop in order to reach or exceed the supercritical temperature of carbon dioxide.

16. The method according to claim 15, wherein each additive among the at least one chemical additive is added by an additional pump that is separate from the pump provided in the loop, and wherein at least one of the following ratios is predetermined:
a first ratio between a total volume of additive delivered by the additional pump and a mass of the tissue to be treated, the first ratio, expressed in ml/g, being between 0.01 and 1; and
a second ratio between a volume flow rate delivered by the additional pump and a volume flow rate delivered by the pump provided in the loop, the second ratio being between 1/5 and 1/100.

17. The method according to claim 14, comprising:
circulating the solvent and co-solvent in a closed circuit in a circuit composed of the reactor and the loop, by opening valves allowing recirculation in the loop and enabling the reactor to remain pressurized, the reactor being functionally coupled, at the inlet, to a valve for reinserting the flow recirculated via the loop, into the internal volume;
and wherein the at least one chemical additive, coming from a tank, is inserted into a section of the loop where carbon dioxide in the supercritical state is circulating towards the inlet.

18. The method according to claim 14, wherein each chemical additive is added into an injection line which joins a communicating path in communication with the area of contact with the tissue, such that each chemical additive circulates in the communicating path as a co-solvent of the carbon dioxide in the supercritical state which is circulating in the reactor in order to traverse the area of contact,
and wherein said communicating path forms a section of the loop.

19. The method according to claim 18, comprising:
before any addition of a chemical additive, a stabilization phase in which the carbon dioxide circulates in a looping circulation in a circuit formed by the reactor and the loop, while pressure in the reactor has reached or exceeded a predefined threshold and a temperature has reached a target temperature range, so as to maintain the carbon dioxide in the supercritical state; and
during said addition, the looping circulation of the carbon dioxide in said circuit is maintained.

20. The method according to claim 14, further comprising implementing, in a pressurized state of the reactor, at least one chemical treatment in the area of contact, which is carried out in an operating mode of the reactor involving said recirculation, by using the loop which is connected to the inlet and the outlet of the reactor, whereby a chemical additive is:
inserted, by using a co-solvent pump and in an open state of an additive insertion valve connected to the loop, into a section of the loop where the carbon dioxide is circulating in the liquid state or in the supercritical state, at a volume flow rate which is at least ten times lower than that of the carbon dioxide circulating in the loop; and
during a supplemental recirculation without addition, recirculated in the reactor and the loop, in a closed state of the additive insertion valve.

21. The method according to claim 20, wherein, between two chemical treatments combining supercritical carbon dioxide and co-solvent, without moving the tissue and without stopping a pump for circulating carbon dioxide in the reactor and in the loop, the following is provided:
a/activating a separation stage connected so as to bypass a section of the loop and accessible by opening a bypass valve and closing a primary valve located downstream of the pressure-regulating valve;
b/allowing the co-solvent and remaining residues to accumulate in the separation stage;
c/purging the co-solvent and remaining residues, through valves of the separation stage; and
d/deactivating the separation stage by closing the bypass valve and opening said primary valve and then restarting treatment with a second co-solvent.

22. The method according to claim 20, wherein, between two chemical treatments combining supercritical carbon dioxide and co-solvent, without moving the tissue, the following is provided:
a/stopping a pump for circulating carbon dioxide in the reactor and in the loop, depressurizing the reactor and the loop;
b/draining the reactor, using a lower drain port of the reactor which forms said outlet, and draining the loop; and
c/purging the reactor and the loop with compressed air.

23. The method according to claim 22, wherein after purging the reactor and the loop with compressed air, the method comprises:
d/rinsing the reactor with purified water by inserting purified water upstream of the reactor and discharging it downstream of the reactor, a direction of circulation of the purified water being reversed.

24. The method according to claim 14, wherein the at least one chemical additive for purification and/or decontamination is a first chemical additive, injected into the loop and inserted into the reactor as a co-solvent of carbon dioxide in the supercritical state, in order to:
carry out a chemical treatment in the area of contact by circulating the first chemical additive in the reactor as well as in the loop which is in communication with said reactor inlet,
the method further comprising, in pressurized state of the reactor:
adding a second chemical additive for purification/decontamination into the loop in order to insert the second chemical additive into the reactor as a co-solvent of carbon dioxide in the supercritical state; and
carrying out a chemical treatment in the area of contact by circulating the second chemical additive for purification/decontamination in the reactor as well as in the loop which is in communication with said reactor inlet, the second chemical additive being reinserted into the reactor via the loop as a co-solvent of carbon dioxide in the supercritical state, during a recirculation with the second additive.

25. The method according to claim 14, wherein a pressure-regulating valve, arranged in the loop downstream of the reactor in a direction of circulation of the treatment flow and upstream of a pump for circulating the supercritical carbon dioxide, is actuated in order to activate one or more pressure drops and increases in the reactor, during the recirculation.

26. The method according to claim 14, wherein recirculation is performed with a filter provided in the loop, so that extraction and separation of a portion of the residues in the loop is allowed.

27. The method according to claim 14, wherein each additive among the at least one chemical additive is added in liquid form.

28. The method according to claim 27, wherein the at least one chemical additive comprises at least two different chemical additives with peracetic acid used as a second chemical additive, after addition of oxygen peroxide as a first chemical additive.

29. The method according to claim 14, wherein carbon dioxide circulating in the loop is cooled by a condenser to reach the liquid state, and wherein the at least one chemical additive is inserted between an outlet of the condenser used to liquefy the carbon dioxide and an inlet of the reactor.

30. The method according to claim 14, wherein the pressure-regulating valve consists of a backpressure regulator included in a discharge line extending from the outlet of the reactor,
and wherein gas flow leaving the reactor is limited by the backpressure regulator so that:
a desired level of pressurization in the reactor is maintained; and
the discharge line circulates CO₂ in the supercritical state between a reactor outlet and the backpressure regulator.

31. A system for treating a tissue or tissue matrix of human or animal origin and in particular based on collagen, by a flow of carbon dioxide in a supercritical state, in order to implement the method according to claim 14, the system comprising:
a reactor provided with an inlet and an outlet, delimiting an internal volume for receiving the tissue, the reactor being adapted to be closed, pressurized, and heated in order to maintain the supercritical state of the carbon dioxide flow;
a pump and a heating device, designed and arranged to cause the carbon dioxide to transition from the liquid state to the supercritical state, upstream of the inlet of the reactor in a direction of circulation going from the pump to the inlet;
a condenser;
a circuit comprising a loop passing through a point or zone for inserting carbon dioxide in the liquid state, the loop extending between a first loop end connected to the outlet of the reactor and a second loop end connected to the inlet of the reactor, in order to allow recirculation of fluid coming from the reactor, from the outlet to the inlet, the pump being arranged in the loop between the first and second ends and downstream of the condenser that allows liquefying carbon dioxide which forms part of recirculated fluid;
a connection device for fluidic connection with the loop and associated with an additive insertion part, to allow adding at least one chemical additive for purification and/or decontamination of the tissue in a section of the loop located downstream of the condenser and upstream of the inlet, such that the connection device forms a mixer enabling a mixed flow, combining the carbon dioxide in the supercritical state as a solvent and the additive as a co-solvent, constituting a treatment flow coming from the loop and reaching the tissue within the internal volume; and
an opening/closing assembly provided with valves arranged on the loop, said assembly being adapted to be configured in a first open state, in which a pressure-regulating device which is part of said valves allows pressurization of the reactor to be maintained above a threshold that is higher than the critical pressure of carbon dioxide in order to activate said recirculation of fluid passing through the loop, while contributing to making the treatment flow dynamic;
the opening/closing assembly also being adapted to be configured:
in a second state that is compatible with an activation of a separation stage without depressurization,
or in a third state that is compatible with a depressurization of the reactor and of the loop,
in order to discharge the co-solvent and residues which are liquid and/or solid, formed during treatment of the tissue and remaining within the internal volume and in the loop.

32. The system according to claim 31, wherein the loop is provided:
with a filter arranged at the outlet of the reactor in order to retain solid residues; and
with a separation stage for separating out the co-solvent and residues discharged from the reactor, which are residues circulating in the loop,
and wherein the pressure-regulating device is arranged in the loop between the filter and two parallel sections of the loop, one of these sections including the separation stage.

33. The system according to claim 31, wherein the pressure-regulating valve consists of a backpressure regulator included in a discharge line extending from the outlet of the reactor, the backpressure regulator allowing pressurization of the reactor to be maintained above 100 bar.
